(19) United States
(12) Patent Application Publication
(10) Pub. No.: US 2006/0258932 A1
(43) Pub. Date: Nov. 16, 2006

(54) Safe motion enabling sequence and system for a medical imaging apparatus

(75) Inventor: Rajagopal Narayanasamy, Bangalore (IN)

Correspondence Address: INTERNATIONAL PATENT COUNSELORS, P.O. BOX 2843, SPOKANE, WA, 99220-2843, US

(73) Assignee: General Electric Company, Schenectady, NY (US)

(21) Appl. No.: 11/125,469

(22) Filed: May 10, 2005

(51) Int. Cl. A61B 5/05

(52) U.S. Cl. 600/407

(57) ABSTRACT

In an embodiment, a safe motion enabling method for a positioner e.g. a vascular positioner in a medical imaging apparatus, includes the actions of (i) holding a positioner at a predetermined stationary position against influence of gravity, (ii) operating a drive motor for the positioner at predetermined low speed; (iii) measuring current drawn by the drive motor and (iv) releasing the positioner in response to the magnitude of the current drawn by the drive motor.

### FIELD OF THE INVENTION

This invention relates generally to, safe motion enabling sequences, and more particularly, to a safe motion enabling sequence and system for a positioner in a medical imaging apparatus.

### BACKGROUND OF THE INVENTION

Generally, a positioner in a medical imaging apparatus is used for positioning of a patient for medical imaging. One example of a positioner is a vascular gantry comprising a C-arm and a pivot axis. Examples of medical imaging apparatus include an X-ray apparatus and a vascular imaging apparatus. The positioner includes mechanisms for lift and pivot in a vascular gantry and longitudinal and lateral tilt in a patient table.

Typically, these mechanisms include one or more drive motors for driving the positioner along various axes e.g. longitudinal, lift and tilt axes, and a brake to hold the positioner in desired state for patient positioning. A motion controller is provided to operate the drive motor in response to a command signal from a central processing unit.

However, movement along the axes such as, for example, lift axis in a vascular gantry, longitudinal axis (in a tilted position) in a patient table are susceptible to influence of gravity and hence require a proper drive sequence for the drive motor for enabling controlled movement of the positioner and hence safe positioning of the patient for medical imaging.

Known systems of drive motor control sequence include releasing the brake after enabling the operation of the drive motor for the axis that is susceptible to influence of gravity. However, although these known systems provide a substantially controlled motion to the positioner, these systems do not allow for a sufficiently safe patient positioning during circumstances such as malfunctioning of the drive motor, failure of the accessories like power amplifier, cable harness, etc.

Thus, there exists a need for a drove motor control sequence wherein the sequence would enable sufficiently safe patient positioning during circumstances such as malfunctioning of the drive motor, failure of the accessories like power amplifier, cable harness, etc

### SUMMARY OF THE INVENTION

The above-mentioned shortcomings, disadvantages and problems are addressed herein which will be understood by reading and understanding the following specification.

In one embodiment, a safe motion enabling sequence for a positioner e.g. a vascular positioner in a medical imaging apparatus, includes the actions of (i) holding the positioner at a predetermined stationary position against influence of gravity, (ii) operating a drive motor for the positioner at predetermined low speed; (iii) measuring current drawn by the drive motor and (iv) releasing the positioner in response to the magnitude of the current drawn by the drive motor.

In another embodiment, a safe motion enabling system for a positioner e.g. a vascular positioner for a medical imaging apparatus includes (i) a motion controller coupled to a drive motor; (ii) a current sensor coupled to the motion controller and the drive motor; (iii) a processor coupled to the current sensor and the motion controller; and a brake couple to the motion controller, wherein the motion controller is configured to operate the brake in response to an output of the current sensor.

In yet another embodiment, a safe motion enabling system, for a positioner e.g. a vascular positioner in a medical imaging apparatus includes (i) a first unit configured to hold the positioner against influence of gravity, (ii) a second unit configured to operate a drive motor for the positioner at predetermined low speed, (iii) a current sensor configured to measure the current drawn by the drive motor and (iv) a processor configured to release the positioner in response to the current drawn by the drive motor.

Apparatus, systems, and methods of varying scope are described herein. In addition to the aspects and advantages described in this summary, further aspects and advantages will become apparent by reference to the drawings and by reading the detailed description that follows.

### BRIEF DESCRIPTION OF THE DRAWINGS

FIG. 1 shows a perspective view of a patient bed as an example of a positioner according to one embodiment of the present invention;

FIG. 2 shows a bottom perspective view of a patient bed of FIG. 1;

FIG. 3 shows an example of a drive control circuit according to this invention;

FIG. 4 shows an example of a servo control loop according to the present invention;

FIG. 5 shows a flow chart of the drive control method according to one embodiment of the present invention; and

FIG. 6 shows an example of a timing diagram for the safe motion enable sequence according to the present invention.

### DETAILED DESCRIPTION OF THE INVENTION

In the following detailed description, reference is made to the accompanying drawings that form a part hereof, and in which is shown by way of illustration specific embodiments which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

Various embodiments of this invention provide a safe motion enabling sequence and system for a positioner e.g. a vascular positioner in a medical imaging apparatus such as, for example, an X-ray apparatus, CT scanner, vascular imaging apparatus, etc

In various embodiments, the safe motion enabling system for a positioner e.g. a vascular positioner driven by a drive motor, includes a first unit configured to hold the positioner at a predetermined stationary position against influence of gravity, a second unit to operate the drive motor at predetermined low speed, a current sensor configured to measure the current drawn by the drive motor, and a processor configured to release the positioner in response to the current drawn by the drive motor.

In an embodiment, the positioner includes a vascular positioner comprising at least one of a vascular gantry and a patient table. For example, the vascular gantry includes at least one lift axis that is susceptible to influence of gravity. In an embodiment, the patient table includes at least one longitudinal axis susceptible to influence of gravity.

For example, in tilted position, longitudinal axis of the patient table is susceptible to influence of gravity.

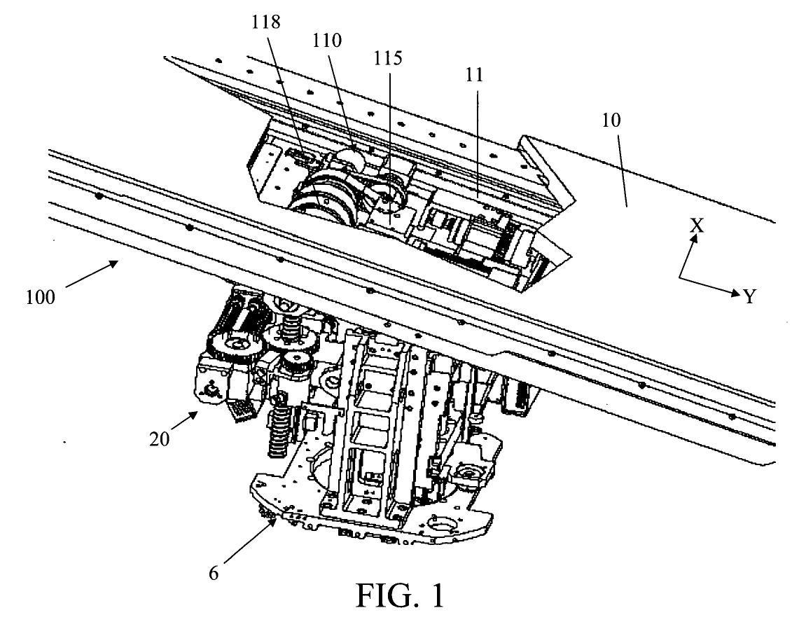

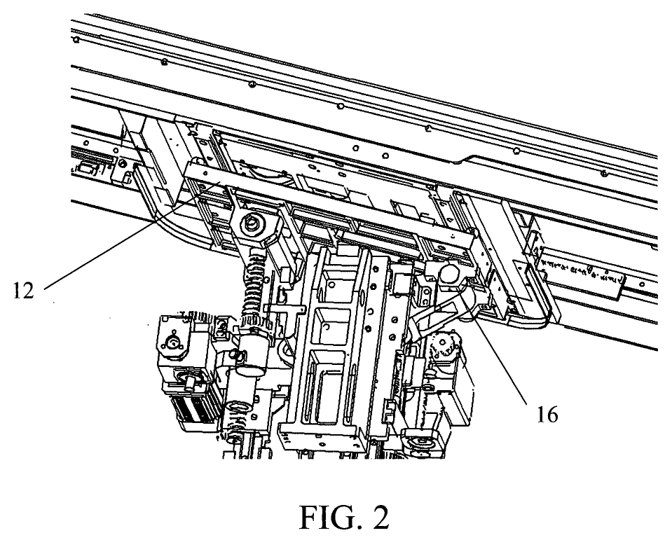

FIG. 1 and FIG. 2 show an embodiment of a patient table comprising a patient bed 100, wherein the patient bed 100 includes at least one patient support surface 10 for supporting a patient for examination. The patient support surface 10 is rigidly coupled to a longitudinal plate 11 from the underside of the patient support surface 10. The longitudinal plate 11 and the patient support surface 10 are movably supported over a tilt plate 12 (see FIG. 2) through a linear bearing (not shown).

In an example, the linear bearing may include a linear block mounted on to the tilt plate and a guide member mounted on to the longitudinal plate.

In an embodiment, the tilt plate 12 is mounted on to a base 6 through a hinge 16 (see FIG. 2). A tilt drive 20 is mounted on the longitudinal plate 11 such that when the tilt drive 20 is actuated, the tilt plate 12 tilts to a predetermined angle about the hinge 16, thereby resulting in tilting movement of the patient support surface 10 relative to ground for convenient patient positioning for examination. In an embodiment at least one longitudinal drive 110 is mounted on the longitudinal plate 11 for moving the patient support surface 10 along a longitudinal axis (Y) e.g. longitudinal direction of the patient support surface 10. The longitudinal drive 110 includes a drive motor 112 (longitudinal drive motor) coupled to the patient support surface 10 through a transmission 115 comprising e.g. a gearbox and a clutch.

For example, the drive motor 112 may be a brushless DC motor.

A brake 118 e.g. an electromagnetic brake is provided in combination with the longitudinal drive 110 to hold the longitudinal plate 11 rigidly when the drive motor 112 is switched OFF.

It should be noted that during patient positioning, in tilted position of the patient bed 100, the brake 118 holds the longitudinal plate 11 at desired position set by the operator, thereby preventing slippage of the patient bed 100 (along the longitudinal axis) due to influence of gravity and hence enable safe patient positioning.

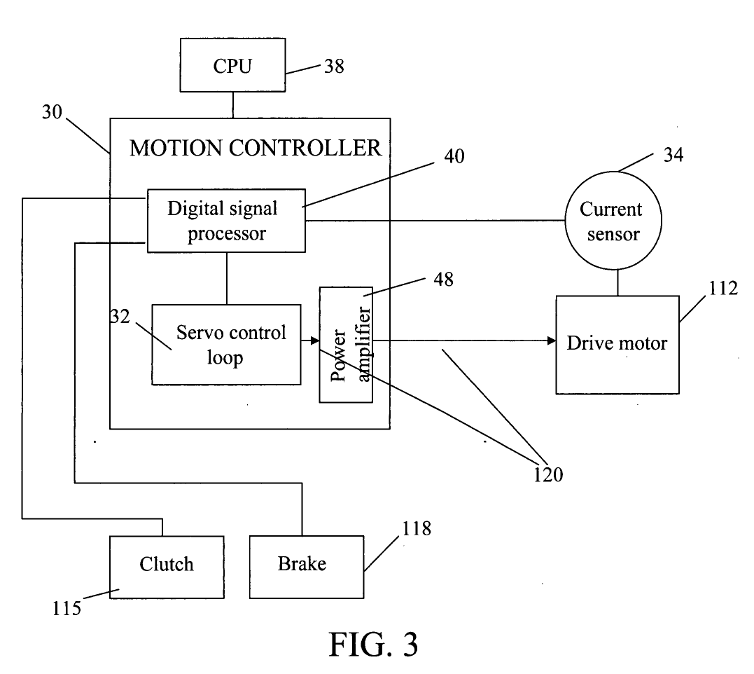

FIG. 3 shows an example of a safe motion enabling circuit according to this invention, wherein the circuit comprises a motion controller 30 configured having a servo control loop 32. A drive motor 112 is coupled to the motion controller 30. A current sensor 34 is coupled to the drive motor 112 and the motion controller 30. The motion controller 30 is coupled to the brake 118.

In an embodiment, a CPU 38 is coupled to the motion controller 30. The CPU 38 is configured to issue a move command to the motion controller 30. For example, the drive motor 112 is a brushless DC motor and the current sensor 34 includes at least one of a current to voltage converter, Hall effect current sensor and a phase current sensor.

In an embodiment, the motion controller 30 includes a digital signal processor 40 implemented with the servo control loop 32.

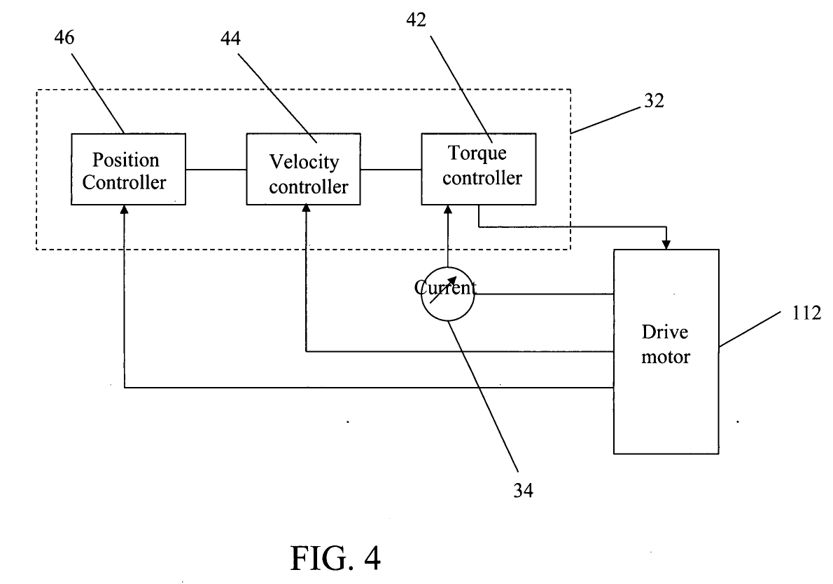

FIG. 4 shows an embodiment wherein, the servo control loop 32 includes at least one of a torque (current) controller 42, a velocity controller 44 and a position controller 46 having at least one of a proportional, integral and derivative (PID) loop configurations.

For example, the current sensor 34 is coupled to the torque control loop. In an embodiment, the torque controller 42, the position controller 46 and the velocity controller 44 are configured to operate at a predetermined low gain.

For example, the values of proportional, integral and derivative gains are set based on the drive motor operating parameters and the drive axes e.g. longitudinal, lateral and tilt axes.

It should be noted that the motion controller 30 may include the central processing unit 38 (CPU) configured within a single module.

It should be noted that other embodiments wherein the central processing unit 38 and the motion controller 30 configured as a separate module are also possible.

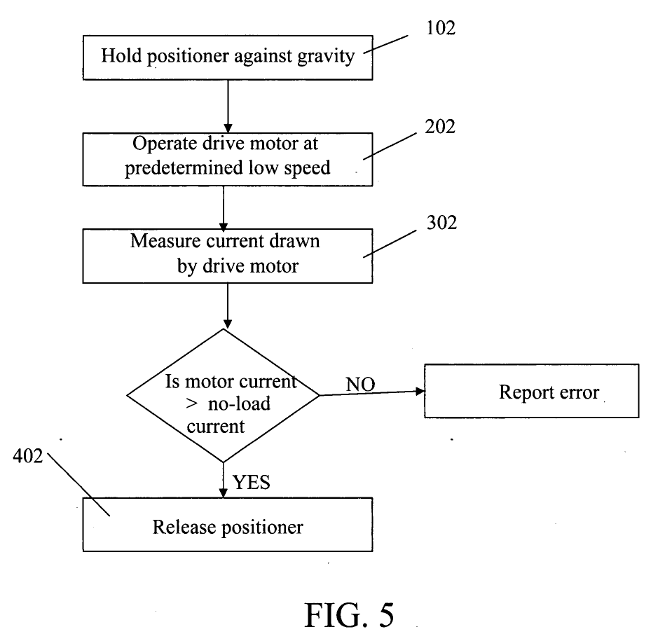

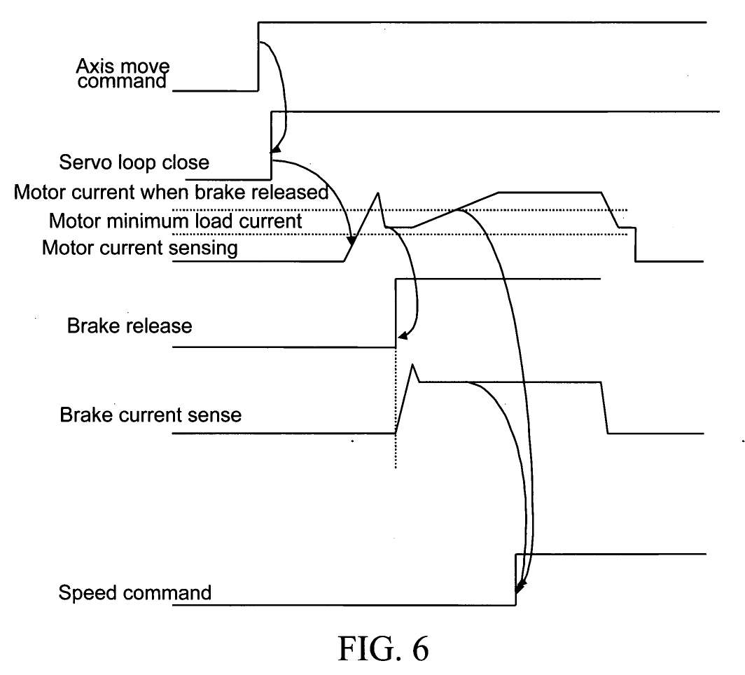

FIG. 5 and FIG. 6 respectively show an example of a flow chart and a timing diagram for a safe motion control sequence for the positioner (patient table), wherein at action 102, the method includes holding the patient bed 100 at predetermined stationary position against influence of gravity.

For example, during patient positioning, the CPU 38 is configured to operate the motion controller 30 to apply brake for rigidly holding the longitudinal plate 11 at a desired (tilted) position.

At action 202, the sequence includes operating the drive motor 112 at a predetermined low speed.

For example, the drive motor 112 is operated at substantially zero speed and further the servo control loop is set at predetermined low gain.

For example, the servo control loop gain is set less than half of the required gain value. The required gain value depends on the drive motor parameters and drive axes e.g. longitudinal, lateral and tilt drive axes. The servo control loop gain values include torque controller proportional gain, torque controller integral gain, velocity controller proportional gain and velocity controller integral gain. The motor parameters are defined by the motor winding resistance, motor winding inductance, load inertia, motor inertia, etc.

At action 302, the sequence includes measuring the current drawn by the drive motor 112 while the drive motor 112 is operating at predetermined low speed. For example, the current measurement may be performed using the current sensor 34.

At action 402, the brake 118 is operated to release the patient bed 100 if the measured current is more than no-load current of the drive motor 112. For example, the motion controller 30 is configured to check whether the current drawn by the drive motor 112 is more than the no-load current of the drive motor 112. If the current drawn by the drive motor 112 is more than no-load current, then the motion controller 30 is configured to release the brake 118 and thus allow the drive motor 112 to move the patient bed 100 to a desired position.

It should be noted that if there exists a malfunctioning of the drive motor 112 or a defect in a cable harness 120 (see FIG. 3) between the servo control loop 32 and the drive motor 112, then the servo control loop 32 cannot control the drive motor 112 and hence cannot enable controlled movement of the patient bed 100 for patient positioning.

It should also be noted that if the current drawn by the drive motor 112 is more than the no-load current, then the drive motor 112 is said to operate against the braking force applied to the patient bed 100. This current measurement is used as a positive feedback to ensure that the cable harness 120 between the servo control loop 32 and the drive motor 112 is intact. Also, the current measurement indicated any malfunctioning of the drive motor 112 and accessories such as, a power amplifier 48 connected to the drive motor 112.

If the current drawn by the drive motor 112 is less than or equal to no-load current, then the motion controller 32 is configured to maintain the brake 118 in hold position, resulting in holding of the patient bed 100 in stationary position.

It should be noted that if the current drawn by the drive motor 112 is less than or equal to no-load current, then the drive motor 112 is said to operate without control from the servo control loop 32 and hence there is a likelihood of uncontrolled movement of the patient bed 100 under the influence of gravity, which may cause patient injury.

Various specific embodiments of this invention provide a method and a system for drive control for a positioner e.g. a vascular positioner in a medical imaging apparatus.

Thus, while the invention has been described with various specific embodiments, it will be obvious for a person skilled in the art to practice the invention with modifications. However, all modifications are deemed to be within the spirit of the claims.

What is claimed is:

1. A safe motion enabling method for a positioner, the method comprising:
(i) holding the positioner at a predetermined stationary position against the influence of gravity;
(ii) operating a drive motor for the positioner at a predetermined low speed;
(iii) measuring a current drawn by the drive motor; and
(iv) releasing the positioner in response to the current drawn by the drive motor.

2. The method according to claim 1 wherein the positioner further comprises a vascular positioner having a positioner axis.

3. The method according to claim 2 wherein the vascular positioner further comprises at least one of a vascular gantry and a patient table.

4. The method according to claim 2 wherein the vascular positioner further comprises a vascular gantry having at least one lift axis susceptible to influence of gravity.

5. The method according to claim 2 wherein the vascular positioner further comprises a patient table comprising at least one longitudinal axis susceptible to influence of gravity.

6. The method according to claim 1 wherein the drive motor is coupled to a motion controller configured having a servo control loop, wherein the operating further comprises operating the drive motor at substantially zero speed and setting the servo control loop at predetermined low gain.

7. The method according to claim 1 wherein the releasing further comprises releasing the positioner when the measured current is more than no-load current.

8. The method according to claim 1 wherein the positioner is coupled to a brake, wherein the brake is operated for holding and releasing the positioner against the influence of gravity.

9. A safe motion enabling system for a positioner, comprising:
(i) a motion controller configured having a servo control loop;
(ii) a drive motor coupled to the motion controller;
(iii) a current sensor coupled to the motion controller and the drive motor; and
(iv) a brake coupled to the motion controller, wherein the motion controller is configured to operate the brake in response to an output of the current sensor.

10. The system according to claim 9 further comprises a digital signal processor implemented with the servo control loop.

11. The system according to claim 9 wherein the positioner further comprises a vascular positioner.

12. The system according to claim 11 wherein the vascular positioner further comprises at least one of a vascular gantry and a patient table.

13. The system according to claim 11 wherein the vascular positioner further comprises a vascular gantry including at least one lift axis wherein the drive motor is configured to drive the vascular gantry along the lift axis.

14. The system according to claim 11 wherein the vascular positioner further comprises a patient table comprising a patient bed having at least one longitudinal axis, wherein, the drive motor is configured to drive the patient bed along the longitudinal axis.

15. The system according to claim 9 wherein the servo control loop is configured to have a predetermined low gain.

16. The system according to claim 9 wherein the drive motor further comprises a brushless direct current motor.

17. A safe motion enabling system for a positioner, comprising:
(i) a first unit configured to hold the positioner against influence of gravity;
(ii) a second unit configured to operate a drive motor of the positioner at a predetermined low speed;
(iii) a current sensor configured to measure a current drawn by the drive motor; and
(iv) a motion controller configured to release the positioner in response to the current drawn by the drive motor.

18. The system according to claim 17 wherein the positioner further comprises a vascular positioner having at least one of a vascular gantry and a patient table.

19. The system according to claim 17 wherein the vascular positioner further comprises a vascular gantry having at least one lift axis susceptible to influence of gravity.

20. The system according to claim 17 wherein the vascular positioner further comprises a patient table comprising at least one longitudinal axis susceptible to influence of gravity.
